(19) United States
(12) Patent Application Publication
(10) Pub. No.: US 2006/0198787 A1
(43) Pub. Date: Sep. 7, 2006

(54) Biomarkers for predicting liver fibrosis treatment efficacy

(76) Inventor: Larry Blatt, San Francisco, CA (US)

Correspondence Address: FOLEY & LARDNER LLP, P.O. BOX 80278, SAN DIEGO, CA, 92138-0278, US

(21) Appl. No.: 11/356,634

(22) Filed: Feb. 17, 2006

Related U.S. Application Data
(60) Provisional application No. 60/654,166, filed on Feb. 18, 2005.

(51) Int. Cl. A61K 49/00; G06F 19/00; A61K 38/21

(52) U.S. Cl. 424/9.1; 424/85.6; 702/19

(57) ABSTRACT

The invention relates to methods for predicting or determining the efficacy of certain medical treatments, especially treatments for liver fibrosis. The methods of the invention include measuring interferon-induced ligands prior to initiating treatment and at some time following the initiation of treatment to predict the clinical outcome of the treatment.

### CROSS-REFERENCES TO RELATED APPLICATIONS

This application claims priority to U.S. Provisional Application No. 60/654,166, filed Feb. 18, 2005, the entire contents of which are herein incorporated by reference and for all purposes.

### FIELD OF THE INVENTION

The present invention is in the field of clinical medicine and relates to methods for predicting or determining the efficacy of certain medical treatments, especially treatments for liver fibrosis. The methods of the invention include measuring interferon-induced ligands prior to initiating treatment and at some time following the initiation of treatment to predict the clinical outcome of the treatment. The invention thus has applications to the field of medicine.

### BACKGROUND

Liver fibrosis is the abnormal accumulation of fibrous scar tissue in the liver. It is a dynamic process resulting from reiterative tissue injury and the chronic activation of tissue repair mechanisms. Fibrosis may be a step in the progression from hepatic inflammation, to fibrosis, to cirrhosis, and even to hepatocellular carcinoma. However, the term fibrosis is often used interchangeably with cirrhosis.

Liver fibrosis has a number of known causes, including but not limited to, parasitic infection, trauma, and autoimmune diseases. Parasitic infection includes both extracellular parasites (e.g., Shistosomes, Clonochis, Fasciola, Opisthorchis, and Dicrocoelium), and intracellular parasites (e.g., viruses, fungi, and even some bacteria). Viruses known to cause liver fibrosis include, but are not limited to, hepatitis A, B, and C, hepatitis delta and epsilon virus, and other viruses that are trophic for hepatic cells.

A major cause of liver fibrosis is hepatitis C virus (HCV), which is estimated to affect about 170 million people worldwide, including 5 million in Western Europe and 2.7-4 million people in the United States (Vrolijk et al. (2004) Netherlands J. Med. 62:76-82; Saadeh and Davis (2004) Cleveland Clinic J. of Med. 71:S3-S7; Foster (2003) Expert Opin. Pharmacother. 4:685-691). The prevalence of HCV varies from 0.5%-2% in most developed countries but is as high as 20% in Egypt (Foster, supra).

70-80% of those infected by HCV develop chronic infections, of which about one-quarter are at risk of developing severe fibrosis (i.e., cirrhosis) within 20 years and half within 50 years. The remaining half of chronically infected individuals remain relatively asymptomatic (Schuppan et al. (2003) Cell Death and Differentiation 10:S59-S67; Patel and McHutchison (2003) Chronic Hepatitis C 114:48-62).

A primary mode of transmission for HCV is intravenous (IV) recreational drug use. The sharing of injection equipment was commonplace 25-30 years ago, before IV drug users were aware of the risk of disease transmission and clean needle programs were available. Accordingly, large numbers of individuals now infected with HCV (and either asymptomatic or suffering only mild, non-descript symptoms) are expected to enter the late stages of HCV infection, sparking fears that they will inundate national healthcare systems with liver cirrhosis cases (Foster, supra).

At present, there are few effective treatments for hepatitis. Treatment of hepatitis resulting from autoimmune disease is generally limited to immunosuppression with corticosteroids. Treatment of viral hepatitis, i.e., caused by hepatitis B and C virus (HBV and HCV, respectively), usually comprises administration of recombinant interferon alpha (IFN-α), optionally with the nucleoside analog ribavirin.

However, treatments involving IFN-α are only 25-50% effective and not well-tolerated by patients. Most complain of flu-like symptoms, including high fever, malaise, fatigue, nausea, and vomiting. Suicidal depression has even been observed in a small number of patients. Clinically, patients treated with IFN-α present with such side-effects as thrombocytopenia, leukopenia, and hemolytic anemia. The addition of ribavirin to the treatment regimen only increases the incidence and severity of side effects. In view of the side effects and the low cure rate, up to 20% of patients terminate treatment, opting to submit to the natural course of the disease, or await more tolerable treatments methods (Foster, supra).

The treatment of HCV infection with IFN-γ, or IFN-γ and ribavirin, has also been investigated, although it is not yet widely used. Unfortunately, IFN-γ treatment causes side effects similar to those associated with IFN-α, forcing patients to make the same difficult decisions regarding treatment.

The CXC Chemokines and Receptors

Chemokines are a subgroup of cytokines that are important in immune and inflammatory response. Chemokines are divided based on the arrangement of conserved cysteine motifs. For example, CC chemokines (β-chemokines) comprise adjacent cysteine residues; CXC chemokines (α-chemokines) comprise cysteine residues separated by a single, additional residue; and CX3C chemokines comprise cysteine residues separated by three additional residues (see, e.g., Cole et al. (1998) J. Exp. Med. 187:2009-2021.

The CXC chemokines are further divided into ELR and non-ELR chemokines, depending on the presence or absence of an additional Glu-Leu-Arg (i.e., ELR) tripeptide sequence adjacent to the CXC motif. Examples of ELR CXCs include interleukin-8 (IL-8), epithelial-derived neutrophil-activating protein (ENA), several growth-related proteins (e.g., GRO-α, β, γ), and neutrophil-activating protein (NAP). Non-ELR CXC chemokines include interferon-γ (IFN-γ)-inducible 10-kDa protein (IP-10), IFN-γ-induced monokine (MIG), IFN-inducible T-cell chemoattractant (iTAC), and stromal cell-derived factor (SDF) (Cole et al.; Sauty et al. (1999) J. Immunol. 162:3549-58.

IP-10, MIG, and iTAC are potent chemoattractants for activated T-cells but not resting T-cells, B-cells or natural killer (NK) cells. Their expression appears to be upregulated in Th1-associated disorders, in response to which IFN-y is expressed. IP-10, MIG, and iTAC expression is primarily associated with activated endothelial cells and IFN-γ-activated macrophages.

The expression of non-ELR CXC chemokines in other cells has also been reported. Specifically, IP-10 is IFN-γ-induced in monocytes, fibroblasts, astrocytes, keratinocytes, neutrophils, and endothelial cells, with expression being associated with, e.g., ulcerative colitis, atherosclerosis, sarcoidosis, tuberculoid leprosy, psoriasis, and viral meningitis (Sauty et al.; Qin et al.). MIG is IFN-γ-induced in peripheral blood mononuclear cells (PBMCs), fibroblasts, keratinocytes, endothelial cells, and PMA-stimulated monocytes. MIG expression is also associated with psoriasis. iTAC is expressed by activated monocytes and astrocytes.

The expression of these non-ELR CXC chemokines would appear to play a role in the recruitment of activated T-cells to the epithelium, likely to promote protective immunity or amplify a Th1-type immune response (Sauty et al.; Qin et al. (1998) J. Clin. Invest. 101:746-54.).

In view of the marginal success rates and significant side effects associated with IFN-based treatments, there exist in the art a need for methods for determining, soon after treatment initiation, whether treatment is likely to be effective. Such methods will allow patients and clinicians to make informed decisions regarding whether to continue with treatments that are causing severe side effects and/or whether to modify treatments that are not providing a therapeutic benefit.

### SUMMARY OF THE INVENTION

The invention provides methods and kits for predicting the therapeutic efficacy of an interferon-based or related method for treating liver fibrosis. The methods include predicting the therapeutic efficacy of the interferon-based treatment based on the change in the level of an interferon-induced ligand in the patient in response to the administration of an interferon to the patient. In certain aspects of the invention, the methods include determining the change in the level of an interferon-induced ligand in the patient in response to the administration of an interferon to the patient; and predicting the therapeutic efficacy of the interferon-based treatment based on the change in the level of the interferon-induced ligand. In related aspects the methods include determining the patient's level of an interferon-induced ligand prior to administration of an interferon to the patient; determining the patient's level of an interferon-induced ligand following commencement of the interferon treatment; and predicting the therapeutic efficacy of the interferon-based treatment based on the change in the level of the interferon-induced ligand. In other related aspects the methods include measuring the patient's level of an interferon-induced ligand, prior to administration of an interferon to the patient; administering a therapeutic amount of an interferon to the patient; measuring the patient's level of the interferon-induced ligand; and determining the change in the level of the interferon-induced ligand in response to the administration of the interferon. One or more interferon-induced ligand may be measured and used according to the method, some of which are identified, herein. Examples of such ligands are iTAC, MIG, and IP-10.

The invention also provides a method for assessing the efficacy of an interferon-based treatment for a liver-related disorder in a patient. In certain aspects the method includes assessing the efficacy of interferon treatment based on the relative increase in the levels of the at least one interferon-induced ligand in the patient. In certain aspects, the method includes measuring a patient's levels of at least one interferon-induced ligand prior to interferon treatment; measuring the patient's levels of the at least one interferon-induced ligand following interferon treatment; and assessing the efficacy of the interferon treatment based on the relative change in the levels of the at least one interferon-induced ligand. In related aspects the method includes determining the relative increase in the levels of at least one interferon-induced ligand in a patient following administration of a composition comprising interferon to the patient; comparing the relative increase to a cut-off value; and assessing the efficacy of interferon treatment based on the relative increase in the levels of the at least one interferon-induced ligand compared to the cut-off value. In other related aspects the method includes measuring a patient's levels of at least one interferon-induced ligand prior to interferon treatment; measuring the patient's levels of the at least one interferon-induced ligand prior following interferon treatment; comparing the relative increase or increases to one or a set of clinically or otherwise determined cut-off or threshold values for each interferon-induced ligand or combinations, thereof; and assessing the efficacy of the interferon treatment based on the relative increase in the levels of the at least one interferon-induced ligand in view of the cut-off value or values. In further related aspects the method includes measuring a patient's levels of at least one interferon-induced ligand, prior to treatment and at least once several weeks following treatment; administering to the patient a therapeutic amount of a composition comprising interferon; determining the relative increase in the levels of the at least one interferon-induced ligand following the administering of the interferon-comprising composition; comparing the relative increase or increases to one or a set of clinically or otherwise determined cut-off or threshold values for each interferon-induced ligand or combinations, thereof; and assessing the efficacy of the interferon treatment based on the relative increase in the levels of the at least one interferon-induced ligand in view of the cut-off value or values.

The invention further provides a method for predicting the clinical outcome of a treatment involving the administration of IFN-γ to a patient with chronic hepatitis. In some embodiments, the method involves measuring the relative change in the serum levels of at least one biomarker, comparing the relative change to cut-off values; and predicting the clinical outcome of the treatment using the information obtained from the comparison and the conclusions or predictions drawn therefrom. Such patients may have or risk developing a liver disorder such as liver fibrosis.

The invention further provides a method for modifying or adjusting a treatment involving the administration of IFN-γ, based on the levels of biomarkers in the patient, the method comprising measuring a patient's serum levels of at least one biomarker prior to treatment and at least once following initiation of treatment, determining the relative change in the levels of at least one biomarker, comparing the relative change to cut-off values, and adjusting the amount of IFN-γ administered to the patient and/or the frequency of administration of IFN-γ, such that the relative change in one of more biomarkers, with respect to the levels prior to treatment, conform to those levels found in patients that have favorable clinical outcomes following IFN-γ treatment.

In a related embodiment, the levels of biomarkers are periodically determined, and the IFN-γ dosage or frequency of administration is adjusted such that a patient receives no more or less IFN-γ that is necessary to keep relative biomarker levels at or near the cut-off values known to be indicative of a favorable clinical outcome.

The invention also provides kits which include parts for practicing the methods described herein and that will be apparent from the examples provided herein. The kit of parts, or kits, may include reagents for collecting and or measuring serum levels of one or more interferon-induced ligand. Such reagents may include antibodies. The kits may further include equipment for collecting and/or processing biological samples. The kits are also likely to contain instructions for use, cut-off values and/or instructions for their determination, and instructions for interpreting the data obtained from the use of the kits.

The invention further provides computer programs and/or algorithms for calculating the relative increase in interferon-induced ligands, determining whether such increases are above or below a threshold level, and/or recommending modifications to a treatment regiment to improve a patient's response to an interferon-based therapy. The computer programs or algorithms may be provided along with necessary hardware, e.g., in the form of a kit or apparatus, which may also accept biological samples and measure the relative levels of intereferon-induced ligands present therein.

### BRIEF DESCRIPTIONS OF THE DRAWINGS

FIG. 1 is a graph showing the change in patient serum iTAC levels, before treatment or at 24 weeks following treatment with IFN-γ (or placebo). IFN-100: 100 μg IFNγ/week; IFN-200: 200 μg IFNγ/week.

FIG. 2 is a graph showing the distribution of patients with >45% increase in serum iTAC levels following treatment with interferon (100 μg/week or 200 μg/week) or placebo. Changes in liver histology are based on Ishak scores.

FIG. 3 is a graph showing changes in liver histology in populations of patients with >45% iTAC or <45% iTAC induction following treatment. Changes in liver histology are based on Ishak scores.

FIG. 4 is a graph showing the change in patient serum MIG levels, before and after treatment with interferon or placebo. IFN-100: 100 μg IFNγ/week; IFN-200: 200 μg IFNγ/week.

FIG. 5 is a graph showing the change in patient serum IP-10 levels, before and after treatment with interferon or placebo. IFN-100: 100 μg IFN-γ/week; IFN-200: 200 μg IFNγ/week.

FIG. 6 shows a logistic regression analysis examining iTAC induction and liver histology outcome.

FIG. 7 shows a logistic regression analysis examining co-variant CXCR3 ligands by liver histology outcome.

FIG. 8 is a table showing the percent change in serum levels of iTAC, MIG, and IP-10 levels, from baseline to week 24, arranged by treatment group.

FIG. 9 is a table showing a logistic regression model for predictors of a stable or improving Ishak fibrosis scores at the end of the treatment study, relative to baseline.

FIG. 10 is a table showing the distribution of patients with ≧59.33% iTAC induction, arranged by treatment group.

FIG. 11 is a table showing the correlation between Ishak fibrosis scores worsening at the end of the treatment study, relative to baseline, and the percent change in iTAC at week 24, relative to baseline.

FIG. 12 is a table showing a logistic regression analysis correlating predictors of a stable or improving Ishak Fibrosis Scores at end of the treatment study, relative to baseline and arranged by treatment group, with observations weighted by percent change in iTAC at Week 24, relative to baseline.

### DETAILED DESCRIPTION OF THE INVENTION

The invention provides materials and methods for predicting the efficacy of liver fibrosis treatment using biological markers that have been determined to be substantially reliable predictors of liver histology. The markers are present in biological samples obtained from the patient. The methods and materials provided by the invention enable the assessment of a patient's treatment progress, thereby delaying or obviating the need for invasive, dangerous liver biopsy and histology to monitor the efficacy of treatment and limiting the suffering of a patient undergoing ineffective therapy.

In one embodiment, the methods and materials of the invention are used to evaluate patients having liver fibrotic diseases, and the biological markers (biomarkers) are CXCR3 ligands that are induced by interferon-γ (IFN-γ). Changes in the levels of these ligands can be detected in the blood following the initiation of an IFN-γ-based treatment. Using the materials and methods of the invention, when the blood serum level of these ligands in a patient having a liver fibrotic disease are measured prior to treatment and at some time following the initiation of a liver fibrosis treatment comprising IFN-γ, the relative increase or decrease of the ligands is indicative of treatment efficacy. The relative change in serum levels is compared to cut-off (or threshold) values as described herein.

Experimental Data and Analysis

The studies that gave rise to the methods of the invention involved the treatment of a population of patients with cirrhotic, chronic hepatitis C, with IFN-γ. The patients were divided into three groups: (1) a group that received a placebo (i.e., no IFN-γ), (2) a group that received 100 μg IFN-γ/week, and (3) a group that received 200 μg IFN-γ/week. Serum samples were collected from patients prior to treatment (designated baseline samples) and at week-24 following the initiation of treatment. The liver histology of these patients was examined at the conclusion of the study, and quantified using Ishak scores (Ishak et al. (1995) Histological grading and staging of chronic hepatitis. J. Hepatol. 22:696-99), which are well-known in the art of diagnosing and treating liver diseases.

The levels of certain CXC cytokines were measured in the collected serum samples. In particular, the levels of the CXCR3 ligands, iTAC, MIG-9, and IP-10, were measured by ELISA assay, using commercially available antibody kits (R&D Systems, Minneapolis, Minn., USA). Statistical analyses of the levels of these CXCR3 ligands were performed to determine whether such ligands were useful biological markers (biomarkers) for the clinical efficacy of IFN-γ treatment, as ultimately determined by improved liver histology.

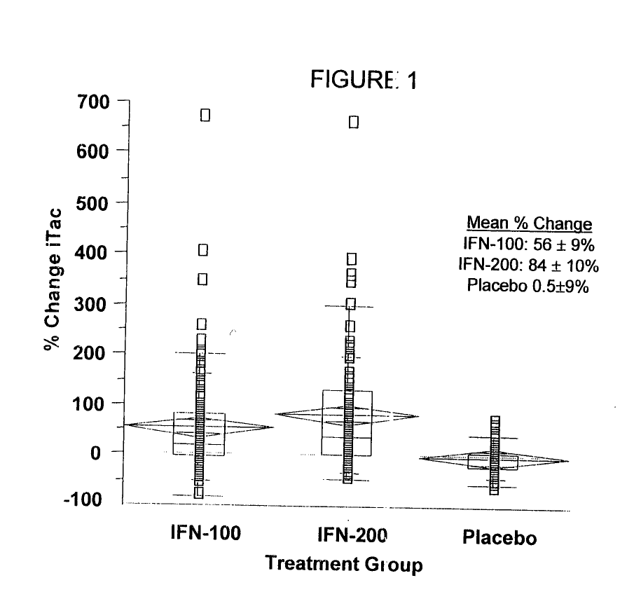

Referring to FIG. 1, patients receiving 100 μg IFN-γ/week (the IFN-100 group) showed a mean increase in serum iTAC levels of about 57%, at 24 weeks following the initiation of treatment compare to baseline levels. Patients receiving 200 μg IFN-γ/week (the IFN-200 group) showed a mean increase in serum iTAC levels of about 84%. Patients receiving placebo showed essentially no increase (0.5%) in serum iTAC levels.

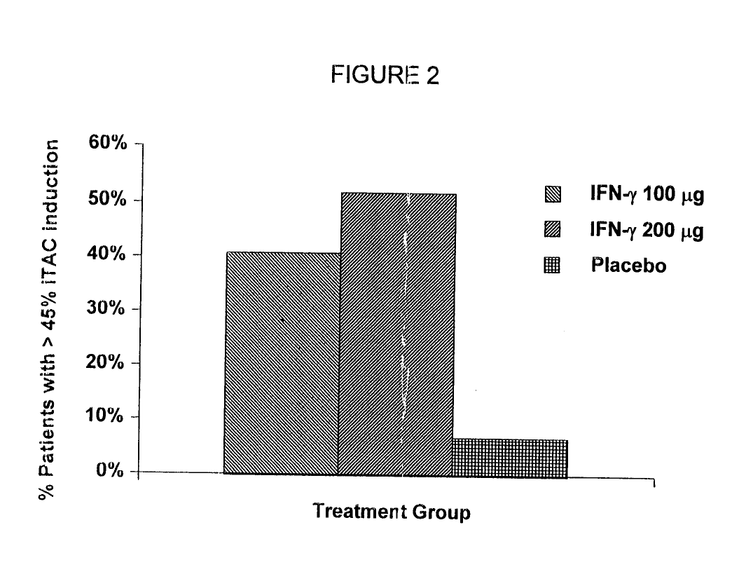

FIG. 2 is a graph showing the distribution of patients with a greater than 45% increase in serum iTAC levels following treatment with interferon or placebo. The results show that increasing iTAC levels are clearly associated with IFN-γ treatment, and that patients receiving a larger dose of IFN-γ show a greater increase in iTAC levels. Less than 10% of patients receiving placebo showed an increase in iTAC levels.

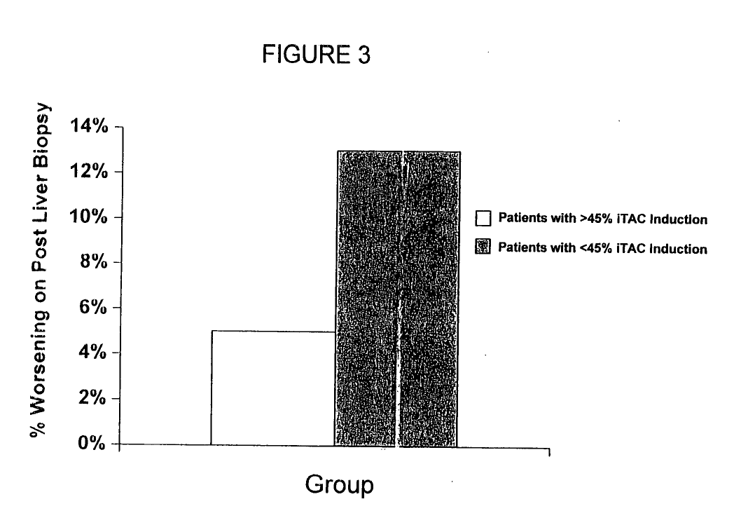

Furthermore, patients with a >45% increase in iTAC levels (also referred to herein as iTAC induction) were less likely to show worsening of liver histology (based on Ishak scores) than patients with a <45% iTAC induction following treatment (FIG. 3). Only about 5% of patients with iTAC induction of >45% showed worsening liver histology, while about 13% of patients with <45% iTAC induction showed worsening liver histology. Note that the more sophisticated statistical analyses performed, below, suggested that an iTAC induction value of about 59% was a better predictor of treatment efficacy than the value of about 45%.

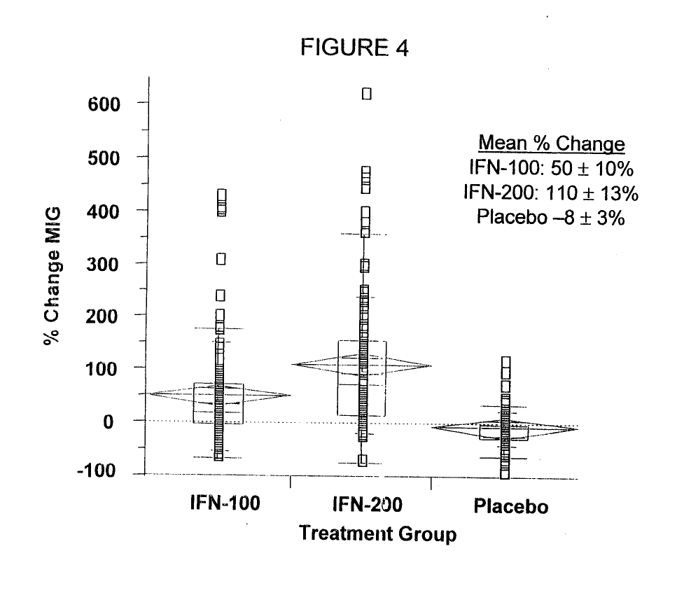

Similar analysis were performed with respect to serum levels of MIG and IP-10. Referring to FIG. 4, the IFN-100 group showed a mean increase in serum MIG levels of about 50%, at 24 weeks following the initiation of treatment compare to baseline levels. The IFN-200 group showed a 110% increase in MIG levels. The placebo group showed essentially no increase (8%).

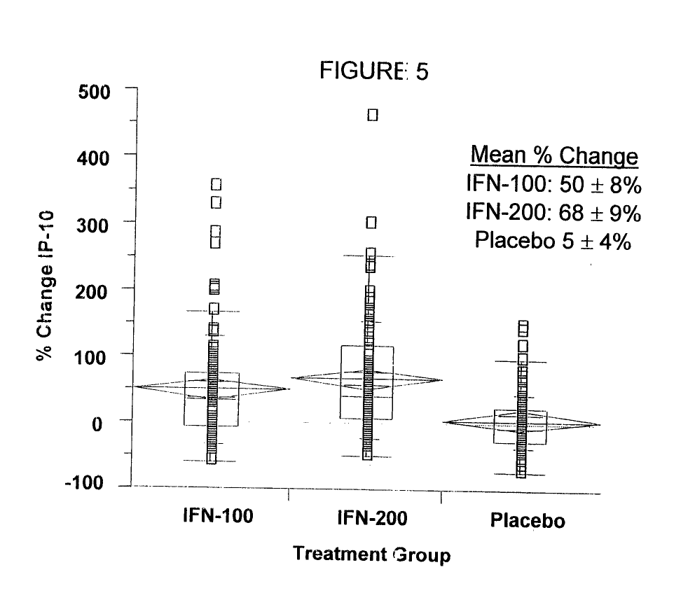

Referring to FIG. 5, the IFN-100 group showed a mean increase in serum IP-10 levels of about 50%, at 24 weeks following the initiation of treatment compare to baseline levels. The IFN-200 group showed a 68% increase and placebo group showed essentially no increase (5%).

These data clearly demonstrated a correlation between an increase in the levels of certain CXCR3 ligands following the initiation of IFN-γ treatment. iTAC induction was found to be a particularly good indicator of the clinical efficacy of treatment. Patients likely to fail treatment could be identified using a cut-off score of from about 45% to about 59%, depending on the statistical analysis used to interpret the data.

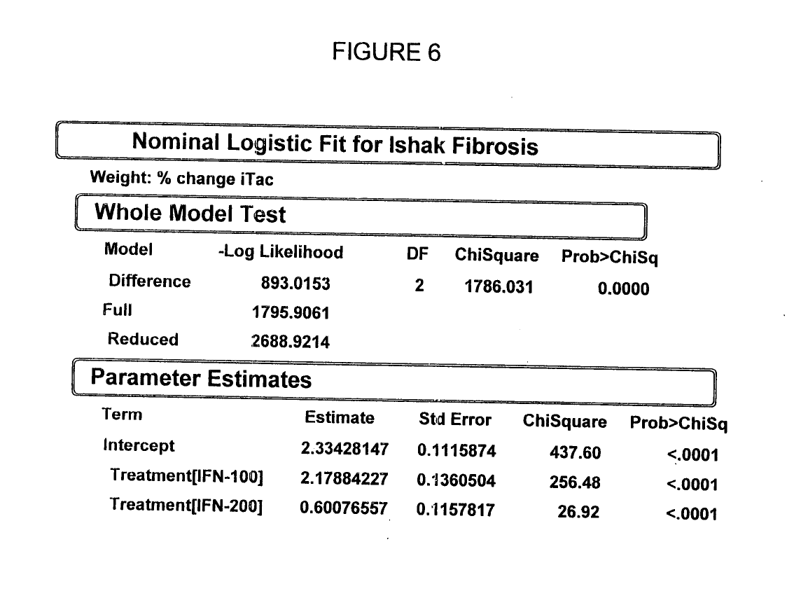

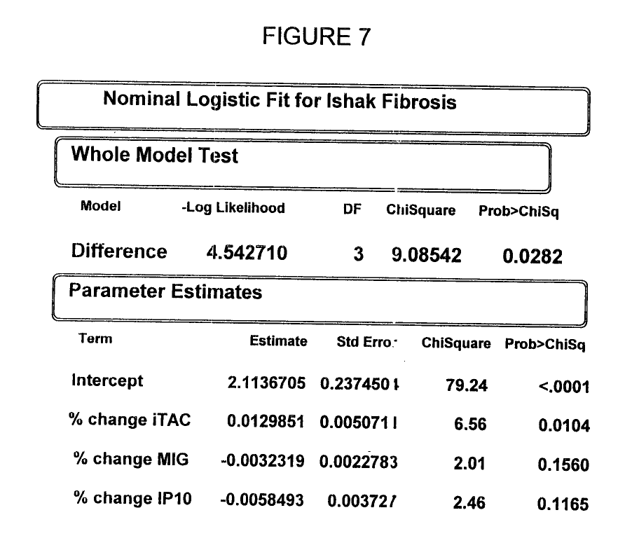

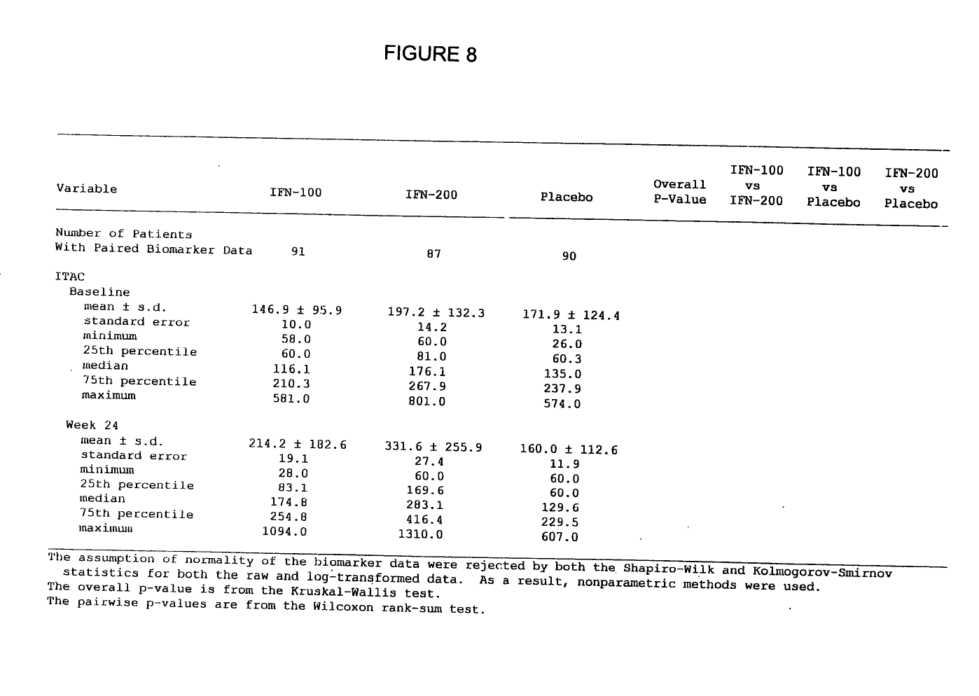

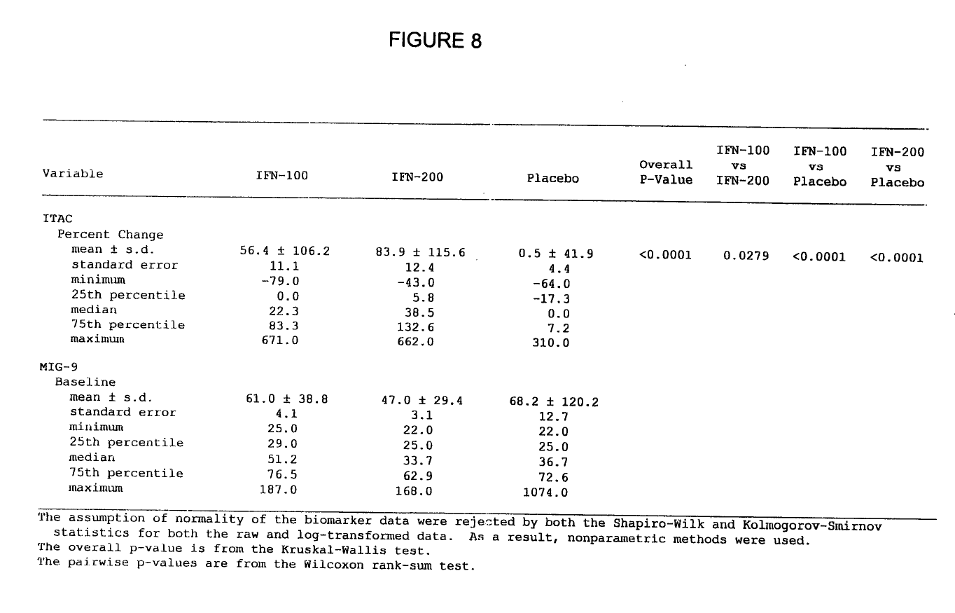

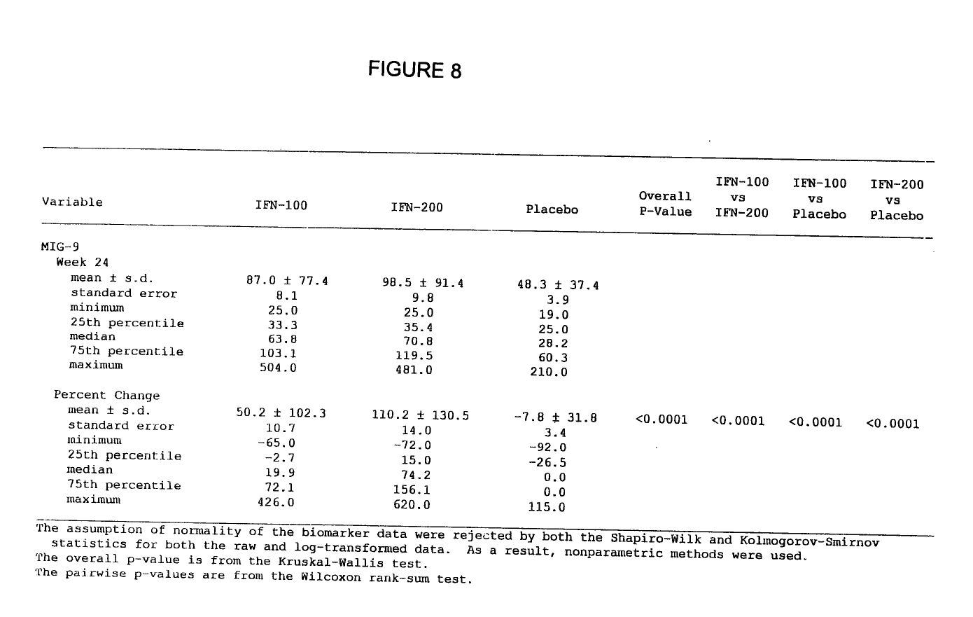

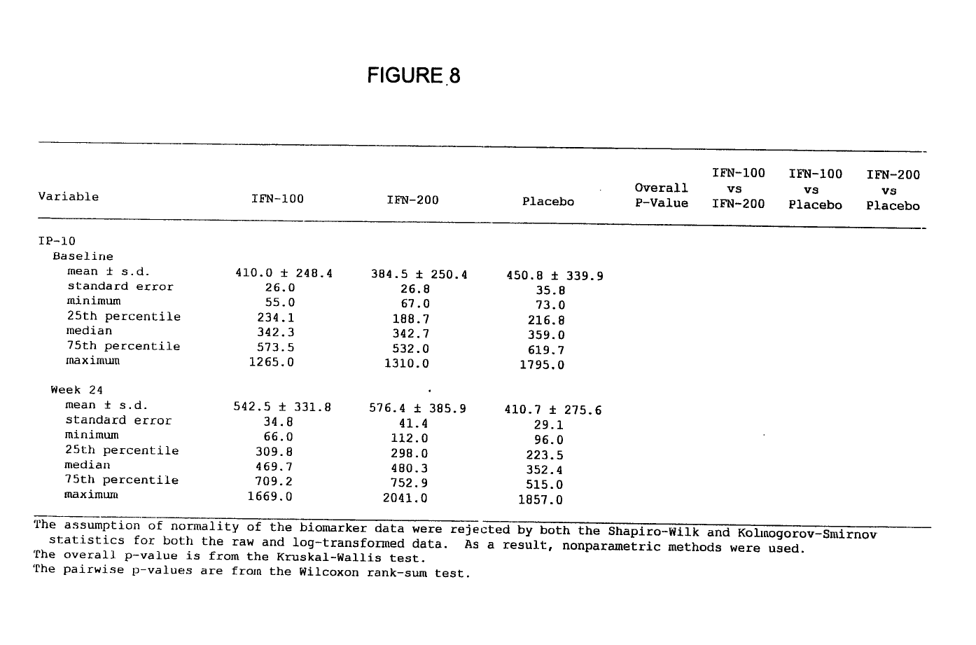

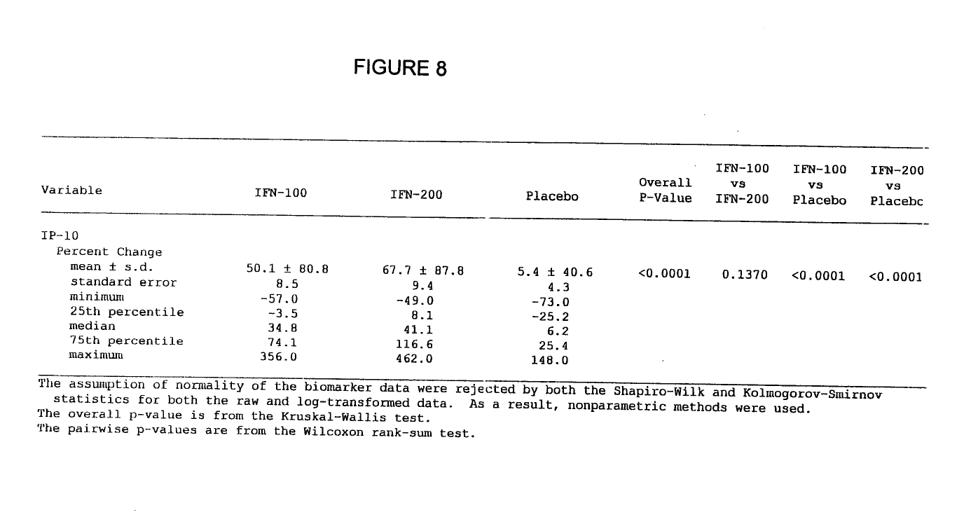

Additional statistical analyses were performed to develop a statistical model that maximized the predictive value of the CXCR3 ligand biomarkers as reliable indicators of treatment efficacy. A logistical regression was performed using the percent change in expression of the biomarkers, from baseline to week 24, on a continuous scale, for predicting the outcome of treatment, which was defined by stable or improving Ishak scores. The levels of the individual biomarkers, (i.e., iTAC, MIG, and IP-10) were included as independent variables in the model, which is shown in FIG. 6. The overall model fit was summarized using log likelihood statistics, and individual parameter estimates were summarized with maximum likelihood estimates and odds ratios, which included Wald chi-square statistics and 95% confidence intervals.

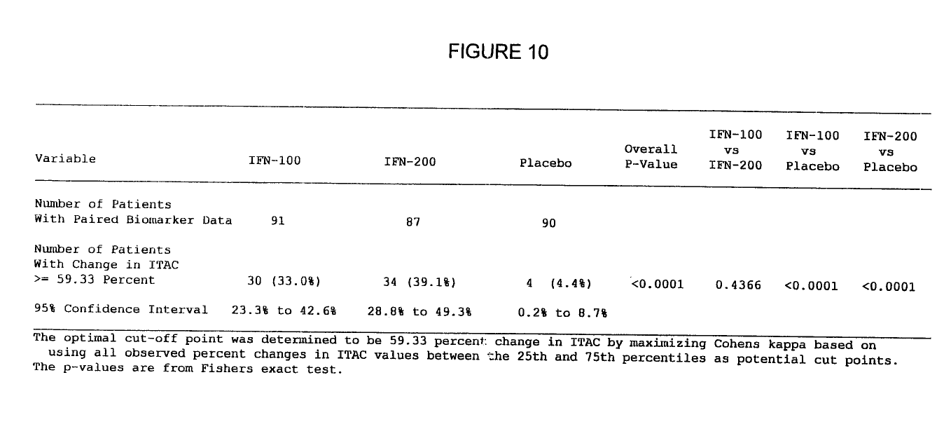

In addition, receiver operating characteristic (ROC) analyses were performed to determine the optimum cut-point (or threshold) for iTAC induction for the purpose of determining whether a patient had stable or improving Ishak fibrosis score. Since iTAC induction follows a continuous numeric scale, the ROC analysis determined the best place to divide the iTAC induction values on a dichotomous scale, such that values on one side of the cut-off would predict stable or improving Ishak fibrosis scores, while values on the other side of the cut-off would predict worsening Ishak fibrosis scores. This cut point was determined by maximizing Cohen's kappa, using all observed percent iTAC induction values between the 25^{th} and 75^{th} percentiles. (FIG. 10). This value was determined to be about 59% (i.e., 59.33%). This value was determined to be 45% using the less comprehensive statistical analyses described above.

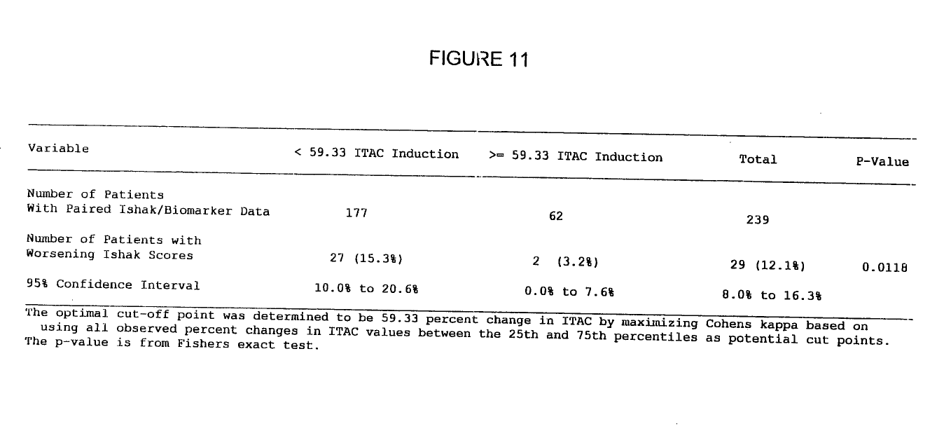

Patients in each treatment group, with percent iTAC induction values at or above this optimal cut-off point, were summarized with counts and percentages. Patients with worsening Ishak fibrosis scores were also summarized with counts and percentages as shown in FIG. 11.

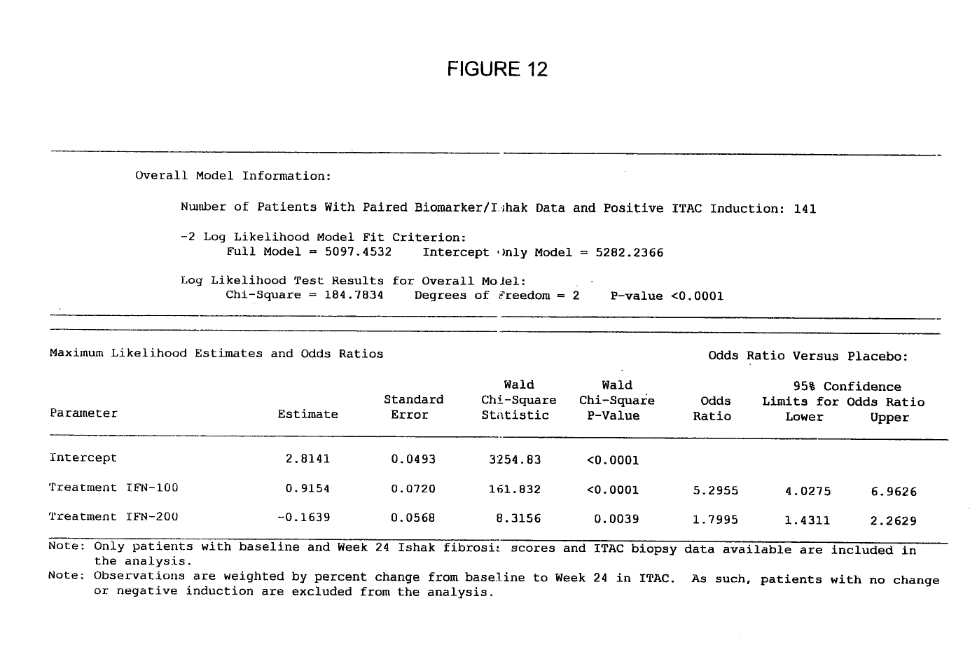

Predictors of Ishak scores remaining the same or improving from baseline to the end of the study were examined using logistic regression (FIG. 12). Treatment was included in the model as the independent variable and data were weighted by percent iTAC induction. Data from patients with higher percent increases of iTAC, relative to baseline, were given more weight than those with a lower percent increases in iTAC. Patients with no change or worsening iTAC were excluded from this analysis. The overall model fit was summarized using log likelihood statistics, and individual parameter estimates were summarized with maximum likelihood estimates and odds ratios, which included Wald chi-square statistics and 95% confidence intervals.

Based on the data and analyses described herein, several correlations were observed. First, patients receiving IFN-γ had significantly greater increases in all biomarkers, compared to patients receiving placebo (P<0.0001 for all observations), demonstrating that the IFN-γ treatment induced the expression of the biomarkers. Secondly, statistical analyses of the percent induction of these biomarkers suggested the existence of substantially discrete patient populations: patients that responded to IFN-γ treatment (“responders”) and patients that did not responds to IFN-γ treatment (“nonresponders”).

Third, using ROC analysis, an iTAC induction cut-off (threshold) of about >59% was established. Patients with about <59% induction of iTAC had significantly worse clinical outcomes than patients with about >59% induction, clinical outcomes being based on liver histology as reported by Ishak scores (P<0.001).

While a goal of the study was to establish cut-off values for biomarker induction that would be reliable in predicting clinical outcome, one skilled in the art will also recognize the related but independent significance of particularly high relative values of biomarker induction. For example, biomarker induction values >200%, are likely to be highly predictive of a successful clinical outcome, notwithstanding the cut-off values. FIGS. 1-3 show that some patients show a 500%, or greater, increase in iTAC, MIG, or IP-10, following treatment with IFN-γ. These high levels of biomarker inductions are simply not observed in patients receiving placebo and are predictive of clinical efficacy. While iTAC appears to be the most reliable biomarker examined in the study, MIG and IP-10 expression also clearly correlate with IFN-γ treatment.

The analyses described herein established reliable cut-off values (thresholds) for interpreting biomarker expression data correlate biomarker expression. This value ranged from 45-59%, depending on the particular statistical analyses performed on the data. However, different cut-off values are likely to be obtained with different patient populations, depending on such factors as the age and overall health of the patients and the existence of other diseases or disorders.

Utility

The methods of the invention can be used to predict the clinical outcome of a treatment comprising the administration of IFN-γ to a patient suffering from liver disease. In one embodiment of the invention, the disease it liver fibrosis, a condition associated with the accumulation of scar tissue in the liver. However, the practitioner will recognize that liver fibrosis is a stage in the progression of various inflammatory hepatic disorders, that lead to fibrosis, cirrhosis and even to carcinoma. Liver fibrosis and cirrhosis are used interchangeably, herein, and the methods are not limited by arbitrarily definitions used by clinicians to identify different stages of liver degeneration.

In addition, numerous scoring systems for measuring liver fibrosis are known in the art, including the Ishak system (used herein), the Scheur system, the Knodell system, and the Metavir system. The methods of the invention may be used with any fibrosis scoring system and is by no means limited to use with only the Ishak system.

The treatment comprising the administration of IFN-γ may further comprise other interferons (including but not limited to IFN-α, β, τ, and ω), specific inhibitors of HCV polypeptides (including but not limited to NS3 and NS5), stress-activated protein kinase (SAPK) inhibitors, pirfenidone and perfinidone analogs, tumor necrosis factor (TNF)-α antagonists, TNF receptor polypeptides or polypeptides that mimic the TNF receptor, antibodies that inhibit TNF-α, thymosin-α, ribavirin, leovirin, viramidine, nucleoside analogs and other antiviral agents. The treatment may also comprise anti-inflammatory agents, antiphychotic or antineurotic drugs, drugs that reduce gastrointestinal discomfort, histamine type 2 receptor antagonists, antacids, antiemetics, antidiarreal agents, hematopoietic agents, and other drugs that have antiviral activity, alleviate the symptoms of viral infection, or alleviate the side-effects of treatment.

According to the methods of the invention, the level of interferon-induced ligands in a biological sample from a patient is determined prior to initiation of therapy (the initial measurement) and at least one time following the initiation of therapy (i.e., the second measurement). The time between the initial and second measurements may be 1 day to about 48 weeks or more (e.g., from about 1 day to about 1 week, from about 1 week to about 2 weeks, from about 2 weeks to about 4 weeks, from about 4 weeks to about 8 weeks, from about 8 weeks to about 12 weeks, from about 12 weeks to about 16 weeks, from about 16 weeks to about 24 weeks, from about 24 weeks to about 48 weeks, or more) after the treatment regimen has been initiated. In a preferred embodiment of the invention, the time interval is about 24 weeks. Similarly, additional measurements (i.e., a third, fourth, fifth, etc. measurement) may be taken at similar time intervals following the second measurement.

The ability to predict the clinical outcome of a treatment, soon after its initiation, will enable clinicians and patients to make informed decisions regarding the course of treatment, including whether to abandon or alter a treatment, e.g., because it is having less beneficial effects than desired or because it is causing further damage to the liver.

The methods will also allow clinicians and patients to monitor the efficacy of treatment over a prolonged period of time, for example, to ensure that a once-effective treatment has not become ineffective or that liver fibrosis has unexpectedly accelerated late in treatment. Such methods may be particularly useful for monitoring the efficacy of liver fibrosis treatment in long-term care patients, patients with liver transplants, or patients having or at risk for autoimmune or immune deficiency disorders, diseases, or conditions.

In one embodiment of the invention, the biomarkers are present in a biological sample obtained from the patient. Biological samples include but are not limited to blood and other liquid samples of biological origin, solid tissue samples, such as a biopsy specimen or tissue cultures or cells derived therefrom and the progeny thereof. The samples may have been manipulated in after procurement, such as by treatment with one or more reagents, solubilization, or enrichment for certain components. Suitable samples for use with the invention include blood, serum, plasma, lymph fluid, synovial fluid, follicular fluid, seminal fluid, amniotic fluid, milk, whole blood, urine, cerebrospinal fluid, saliva, sputum, tears, perspiration, mucus, cell culture medium, cell supernatant, and cell or tissue extracts.

Interferons for use in the invention may be naturally occurring, recombinant, or synthetic. Such interferons may be truncated, substituted, or modified. In one embodiment of the invention, the interferons are pegylated at one or more amino acid residues. The peg moieties may be linear or branched, attached directly or via a linker, and attached at the N-terminus, C-terminus, specific internal amino acid residues, or otherwise attached to one or more of the above-described interferons.

In one embodiment of the invention, a relative increase in the levels of biomarkers, is indicative of a successful clinical outcome for the treatment, i.e., the reduction in liver fibrosis. In a preferred embodiment of the invention, the relative increase in biomarker levels is compared to cut-off values known to reliable in predicting clinical outcome. The iTAC cut-off values described herein range from about 45% to about 59%. However, other cut-off values can be obtained, e.g., for different patient populations, using the experimental and statistical methods disclosed herein. Such cutoff values may be less than 45%, but at least, for example, 40%, 35%, 30%, 25%, or 20%. Such cut-offvalues may also be greater than about 59%, for example, at least about 65%, 75%, 85%, 95%, or more. In preferred embodiments of the invention, the cut-off values are likely to be between about 45% and 65%, for example, 50%, 55%, or 60%.

It may be desirable to determine specific cut-off values for one or more particular groups of patients, for use in combination with, or instead of the cut-off values disclosed herein. Moreover, experience with large patient populations may eventually allow the “fine-tuning” of cut-off values, without departing from the underlying methodologies described herein.

A relative decrease in the biomarker levels, e.g., before and after treatment, is generally indicative of treatment failure or lack of response by the patient, even without the use of cut-off or threshold values.

Monitoring the relative levels of biomarkers throughout, or during at least part of, IFN-γ treatment will also allows patients and clinicians to adjust an IFN-γ treatment regimen for optimal therapeutic benefit. For example, patients with relative increases in biomarkers that are below the cut-off values can be given increased amounts (dosages) of IFN-γ, more frequent administrations of IFN-γ, different forms of IFN-γ, or other pharmaceutical compositions to increase the efficacy of the IFN-γ treatment, including but not limited to those identified above and/or below, to increase their biomarker levels. It may also be desirable to give these patients other pharmacological composition, such as those discussed above and/or below, to reduce the side-effects associated with increased dosages of IFN-γ.

Conversely, it may be desirable to reduce the dosage and/or frequency of IFN-γ administration in patients with high relative levels of biomarkers, such that the patient maintains relative biomarker levels that are at or above the cut-off values, but the patient does not receive more IFN-γ than is necessary to achieve a therapeutic effect. In this manner, side effects from IFN-γ treatment are minimized without sacrificing therapeutic efficacy. Upon reducing the amount of IFN-γ that a patient receives, it may also be desirable to reduce the amount of other pharmaceutical compositions, such as those used to reduce the side-effects associated with IFN-γ treatment. In this manner, a patient receives no more pharmacological agents than is necessary to maintain their biomarker levels with a range that is associated with effective treatment of liver fibrosis.

The levels of biomarkers are measured in biological samples obtained from the patient. In a preferred embodiment, the biological sample is blood or serum, in which case obtaining the samples from a patient is relatively simple and non-invasive procedure. Methods of obtaining blood are well-known in the art are not part of the invention. In addition, numerous methods for detecting and quantifying polypeptides, including the instant biomarkers, are known. Such methods include but are not limited to antibody-based methods. The particular methods of detecting and quantifying the biomarkers are not important to the invention.

The methods of the invention may be combined with other methods for predicting the efficacy of treatment for liver fibrosis. In addition, the methods may be used to monitor the clinical progress or predict the clinical outcome of other diseases associated with CXCR3 ligand expression.

For example, the expression of IP-10, MIG, and iTAC is known to be upregulated in Th1-associated disorders, in response to which IFN-γ is expressed. Th1-mediated disorders include but are not limited to delayed-type hypersensitivity, insulin-dependent diabetes mellitus, multiple sclerosis, rheumatoid arthritis, contact hypersensitivity, and inflammatory bowel disease. Using the biomarkers and statistical methods described herein, one skilled in the art can determine appropriate cut-off values for changes in the serum levels of these biomarkers that correspond to particular disease states in different Th1-mediated disorders. Serum levels of the ligands can then be measured, compared to the cut-off values, and used to monitor disorders, or predict the clinical outcome of disorders, in patients with Th1-mediated disorders, including patients receiving treatment for such disorders.

In this manner, serum IP-10 levels may be particularly important in monitoring/predicting the course of diseases such as ulcerative colitis, atherosclerosis, sarcoidosis, tuberculoid leprosy, psoriasis, and viral meningitis. Serum MIG levels may be particularly important in monitoring/predicting the course of psoriasis.

The levels of biomarkers are also of value in distinguishing between different forms of B-cell lymphomas. For example, MIG, is expressed in some B-cell lymphomas, including B CLL/SLL (Jones et al. (2000) Neoplasia 95:627-632).

The percent change in the level of interferon-inducible gene product can be calculated manually, e.g., by a human, or completely or partially performed by a computer program or algorithm along with necessary hardware, such as input, memory, processing, display, and output devices.

Upon being provided with data relating to a patients levels of interferon-induced ligands prior to interferon administration and following interferon treatment, the computer program or algorithm can calculate the percent increase in the level of interferon-inducible gene product, determine whether the increase is above or below a threshold, assess the efficacy of the treatment regimen, and propose modifications to the therapy intended to increase the likelihood that a patient will respond favorably to treatment.

The instant invention further provides an apparatus for determining the levels of interferon-induced ligands in a biological sample. In a preferred embodiment of the invention, the apparatus is capable of measuring the levels of one or more interferon-induced ligands in a biological sample, storing such data, and ultimately using such data in the analyses described, upon. According to one embodiment of the invention, the apparatus is portable. In one embodiment, the apparatus is suitable for use in a physicians office, providing the physician with the means for quickly determining the efficacy of an interferon-based treatment without sending biological sample to a clinical laboratory. In another embodiment of the invention, part or all of the data collection and analysis may be performed by a clinical laboratory.

The above-described computer programs and/or apparatus are likely to be provided to physicians or clinical laboratories with appropriate instructions and reagents, including antibodies.

### EXAMPLES

The following terms are given the following meanings. All other terms used herein are to be given their ordinary meanings in the relevant art.

| | |
|---|---|
| ELISA | enzyme-linked |
| | immunosorbent assay |
| mg | milligrams |
| μg | micrograms |
| l or L | liter |
| dl or dL | deciliter |
| ml | milliliter |

### Example 1

### Measurement of CXCR3 Biomarker Serum Levels

Serum samples were collected from test subjects/patients prior to treatment (baseline samples) and at week 24 following initiation of therapy. The serum levels, expressed as mg/dL, of each of the three CXCR3 ligands, iTAC, MIG-9, and IP-10, were measured by ELISA assay, using antibodies specific for each ligand.

The ANOVA test was used to determine statistical significance with a post hoc t-test to determine pair-wise differences.

### Example 2

### Percent Change in iTac Levels Following IFN-γ Therapy in Cirrhotic Chronic Hepatitis C Patients

The data obtained from Example 1 were used to calculate the relative change in serum levels of iTac at week 24 following treatment, relative to the baseline levels. The results are shown below:

| | Mean % |
|---|---|
| Group | Change |
| IFN-100 | 56 ± 9 |
| IFN-200 | 84 ± 10 |
| Placebo | 0.5 ± 9 |

These results demonstrated that patients treated with IFN-γ at 100 mg or 200 mg per week had significantly greater percent increase in iTac (about 56% and 84%, respectively) when compared to patients receiving placebo (P<0.0001).

### Example 3

### Percent Change in MIG Levels Following IFN-γ Therapy in Cirrhotic Chronic Hepatitis C Patients

The data obtained from Example 1 were used to calculate the relative change in serum levels of MIG at week 24 following treatment, relative to the baseline levels. The results are shown below:

| | Mean |
|---|---|
| Group | % Change |
| IFN-100 | 50 ± 10 |
| IFN-200 | 110 ± 13 |
| Placebo | 8 ± 3 |

These results demonstrated that patients treated with IFN-γ at 100 mg or 200 mg per week had significantly greater percent increase in MIG (about 50% and 110%, respectively) when compared to patients receiving placebo (P<0.0001).

### Example 4

### Percent Change in IP-10 Levels Following IFN-γ Therapy in Cirrhotic Chronic Hepatitis C Patients

The data obtained from Example 1 were used to calculate the relative change in serum levels of IP-10 at week 24 following treatment, relative to the baseline levels. The results are shown below:

| | Mean |
|---|---|
| Group | % Change |
| IFN-100 | 50 ± 8 |
| IFN-200 | 68 ± 9 |
| Placebo | 5 ± 4 |

These results demonstrated that patients treated with IFN-γ at 100 mg or 200 mg per week had significantly greater percent increase in IP-10 (about 50% and 68%, respectively) when compared to patients receiving placebo (P<0.0001).

### Example 5

### Logistic Regression Model Examining Co Variants CXCR3 Ligands by Liver Histology Outcome (Per/Post Ishak Score)

Table 1 shows the percent change, from baseline to week 24, in the serum levels of iTAC, MIG, and IP-10, organized by treatment group, i.e., patients receiving placebo, 100 μg/week IFN (IFN-100), or 200 μg/week IFN (IFN-200). The number of patients in each group, average percent change with standard deviations, minimum, maximum, and percentile scores are shown.

Statistical analyses were applied to the serum level data obtained for iTAC, MIG, and IP-10 to determine which of these biological markers were reliable predictors of changes in liver histology, as determined using Ishak scores (Ishak et al. (1995) Histological grading and staging of chronic hepatitis. J Hepatol 22:696-99). Initially, the data were found to violate the assumption of normality using both Shapiro-Wilk and Kolmogorov-Smimov statistics. The data were then analyzed using nonparametric tests. Specifically, differences in the serum levels of each of the three CXCR3 ligand were tested using the Kruskal-Wallis test, and pairwise comparisons were performed using the Wilcoxon rank-sum test.

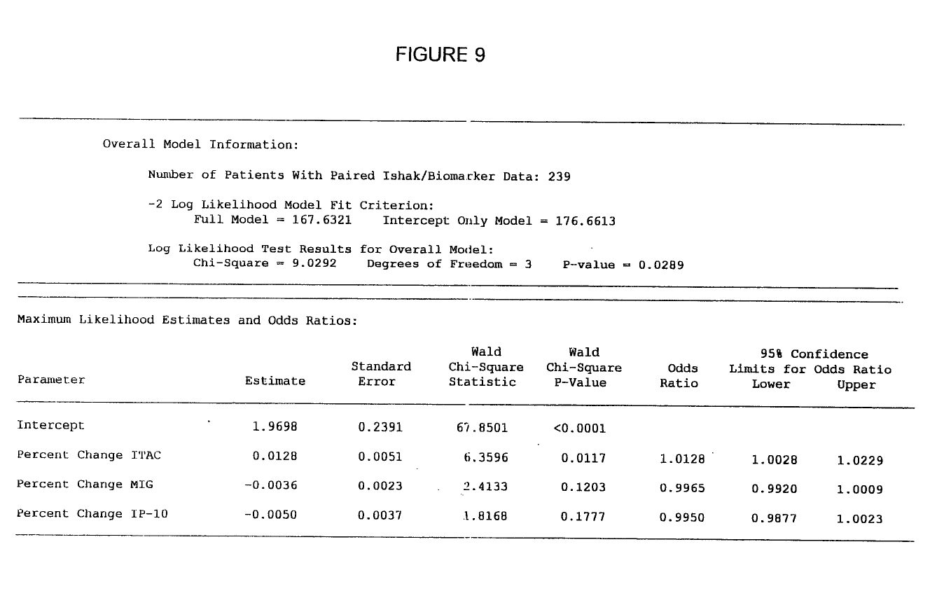

Predictors of Ishak scores that remained the same or improved at week 24, relative to baseline, were examined using logistic regression (FIG. 9). The percent change in expression of iTAC, MIG, and IP-10, from baseline to week 24, were included as independent variables in the model. The overall model fit was summarized using log likelihood statistics, and individual parameter estimates were summarized with maximum likelihood estimates and odds ratios, which included Wald chi-square statistics and 95% confidence intervals.

Receiver operating characteristic (ROC) analyses were performed based on the ability of the “percent iTAC induction,” i.e., the percent change in iTAC levels from baseline to week 24, to determine if patients had stable or improving Ishak fibrosis scores from baseline to end of treatment (FIG. 10). The optimal cut-off point for percent iTAC induction was determined by maximizing Cohen's kappa using all observed percent iTAC induction values between the 25^{th} and 75^{th} percentiles. Patients in each treatment group with percent iTAC induction values at or above the optimal cut-off point were summarized with counts and percentages.

Patients with worsening Ishak fibrosis scores were summarized with counts and percentages based on percent iTAC induction (i.e., patients with percent ITAC induction below the optimal cut-off point versus patients with percent iTAC induction at or above the optimal cut-off point). These data are summarized in table 4.

Predictors of Ishak scores remaining the same or improving from baseline to the end of the study were examined using logistic regression (FIG. 12). Treatment was included in the model as the independent variable and data were weighted by percent iTAC induction. Data from patients with higher percent increases of iTAC, relative to baseline, were given more weight than those with a lower percent increases in iTAC. Patients with no change or worsening iTAC were excluded from this analysis.

The overall model fit was summarized using log likelihood statistics, and individual parameter estimates were summarized with maximum likelihood estimates and odds ratios, which included Wald chi-square statistics and 95% confidence intervals.

The above examples should in no way be construed to limit the invention. Other embodiments and/or uses of the invention will be apparent to those skilled in the art in view of the instant disclosure.

All references identified herein are incorporated into the application in their entirety to the same extent as if each reference was individually incorporated in its entirety.

What is claimed is:

1. A method for predicting the therapeutic efficacy of an interferon-based treatment for liver fibrosis, said method comprising
predicting the therapeutic efficacy of the interferon-based treatment based on the change in the level of an interferon-induced ligand in said patient in response to the administration of an interferon to said patient.

2. The method of claim 1, wherein the interferon-induced ligand belongs to the family of CXCR3 ligands.

3. The method of claim 2, wherein the ligand is selected from the group consisting of iTAC, MIG, and IP10.

4. The method of claim 1, wherein the ligand is iTAC.

5. The method of claim 1, wherein the interferon is IFN-γ.

6. The method of claim 3, wherein an at least about 55% increase in the relative levels of iTAC is predictive of a favorable response to interferon.

7. The method of claim 3, wherein less than an about 55% increase in the relative levels of iTAC is predictive of a disfavorable response to interferon.

8. The method of claim 3, wherein an at least 45% increase in the levels of iTAC is associated with not worsening of liver histology.

9. The method of claim 3, wherein the several weeks of treatment is about 24 weeks.

10. The method of claim 1, wherein the composition comprising interferon further comprises ribavirin.

11. The method of claim 1, wherein the interferon is pegylated.

12. The method of claim 1, further comprising comparing the change in the level of the interferon-induced ligand to a cut-off value, wherein a change in the level that meets or exceeds the cut-off value is indicative or predictive of a favorable clinical outcome and a change in the level that is below the cut-off value is indicative or predictive of an unfavorable clinical outcome.

13. A method for assessing the efficacy of an interferon treatment for a liver disorder in a patient, comprising:
assessing the efficacy of the interferon treatment following administration of a composition comprising interferon to said patient based on the relative increase, compared to a cut-off value, in the levels of at least one interferon-induced ligand.

14. A method for predicting the clinical outcome of a treatment comprising the administration of IFN-γ to a patient with chronic hepatitis the method comprising: measuring the relative change, from a time prior to treatment to a time following the initiation of the treatment, in the serum levels of at least one biomarker selected from the group consisting of iTAC, MIG, and IP-10; comparing the relative change to at least one cut-off value; and predicting the clinical outcome of the treatment using the information obtained, therefrom.
